(19) United States
(12) Patent Application Publication
(10) Pub. No.: US 2024/0218449 A1
(43) Pub. Date: Jul. 4, 2024

(54) METHOD FOR OBTAINING EXTRACELLULAR VESICLES AND USES THEREOF

(71) Applicant: BEKEN BIO, INC., SAN DIEGO, CA (US)

(72) Inventors: PATRIK SIMMLER, SCHAFFHAUSEN (CH); CHRISTOPHER MILLAN, SAN DIEGO, CA (US)

(21) Appl. No.: 18/278,422

(22) PCT Filed: Feb. 22, 2022
(86) PCT No.: PCT/EP2022/054398

§ 371 Date: Aug. 23, 2023

(30) Foreign Application Priority Data
Feb. 23, 2021 (EP) 21158817.3

(51) Int. Cl. C12Q 1/6886; C12N 5/09; G01N 33/574

(52) U.S. Cl. CPC C12Q 1/6886; C12N 5/0693; G01N 33/57484; C12N 2533/74; C12N 2537/10

(57) ABSTRACT

The present invention relates to methods for producing ex-vivo extracellular vesicles from solid cancer cells and uses thereof.

### FIELD OF THE INVENTION

The present invention relates to methods for ex-vivo producing extracellular vesicles from cancer cells and uses thereof.

### BACKGROUND OF THE INVENTION

Extracellular vesicles (EVs), such as exosomes and microvesicles, are released by various cell types and have been identified as mediators of intercellular communication by transmitting specific information from their cell of origin to their target cells. These vesicles are involved in many (patho)physiological processes and increasingly attract attention as novel tools.

In the case of cancers, the sustained growth, invasion, and metastasis of cancer cells depend upon bidirectional cell-cell communication within complex tissue environments. Such communication predominantly involves the secretion of soluble factors by cancer cells and/or stromal cells within the tumour microenvironment (TME). However, these cell types also export membrane-encapsulated particles containing regulatory molecules that contribute to cell-cell communication, those particles are known as extracellular vesicles (EVs) and include species of exosomes and shed microvesicles (Xu et al., 2018, Nat Rev Clin Oncol, 15(10): 617-638).

Extracellular vesicles were also identified as promising tools for various therapeutic approaches (Lener et al., 2015, Journal of Extracellular Vesicles, 4:30087).

Identifying the EV-specific molecular cargo involved in these (patho)physiological pathways could provide novel therapeutic targets or diagnostic biomarkers for widespread clinical application. EVs isolated directly from biofluids of cancer patients would seemingly provide the ideal source for such studies, but the extremely low concentration of tumor-specific EVs in systemic blood relative to EVs from other tissues (Johnsen et al., 2019, Biochim Biophys Acta Rev Cancer, 1871: 109), coupled with the technical challenge of isolating EVs from biofluids at sufficient purity for downstream omics analysis (Ramirez et al., 2018, Nanoscale, 10:881), represent significant challenges. To circumvent this, cancer cells cultured in vitro on 2D tissue culture plastic are often used as EV sources for molecular characterization. However, 2D culture has been shown recently to introduce aberrant cell behaviors including irregular expression of cancer-specific antigens (Weigelt et al., 2014, Adv Drug Deliv Rev, 69-70:42). For example, a number of potential biomarkers identified in EVs released by prostate cancer cells grown in 2D were not identified in follow-up studies analyzing EVs in urine, serum, or plasma of prostate cancer patients (Nawaz et al., 2014, Nat Rev Urol, 11(12):688). Thus, there is a need for methods for obtaining and isolating extracellular vesicles of physiological relevance.

### OBJECTIVES AND SUMMARY OF THE INVENTION

The inventors have realized that three-dimensional cell culture systems of the invention allow to obtain extracellular vesicles that differ in their properties from extracellular vesicles obtained by two-dimensional cell culture systems. In particular, the inventors realized that extracellular vesicles obtained by three-dimensional cell culture systems as described therein are more potent in displaying antigens compared to extracellular vesicles obtained by two-dimensional cell culture systems or other “3D” cell culture environments. Thus, extracellular vesicles obtained by three-dimensional cell culture systems according to the invention are of interest for use in the field of the detection of specific tumor antigens.

A general object of this invention is to provide methods for ex-vivo obtaining extracellular vesicles from cancer cells of solid tumors.

One of the specific objects of this invention is to provide an efficient method for the ex-vivo production of extracellular vesicles in high yields.

It is advantageous to provide a method of growing solid tumor cancer cells under conditions which not only allow creating a 3D environment between cells where each cell senses the neighboring cells such as in a micro-tumoral environment but also which is favorable to the production of specific cancer cell EVs in the culture medium.

It is advantageous to provide a method of growing solid tumor cancer cells under conditions that trigger or enhance phenotypic features of the secreted EVs that would be relevant for a certain type of cancer.

Another object of this invention is to provide ex-vivo generated extracellular vesicles from solid tumor cancer cells, said ex-vivo generated extracellular vesicles expressing tumor specific antigens that are physiologically relevant to a specific type of cancer.

It is advantageous to provide ex-vivo generated extracellular vesicles from cancer cells that would express specific tumor antigens in high yields.

It is advantageous to provide ex-vivo generated extracellular vesicles from cancer cells expressing specific tumor antigens which can be easily used for identifying a selection of specific tumor antigens in view of the development of targeted diagnosis or therapy.

It is advantageous to provide ex-vivo generated extracellular vesicles from cancer cells expressing alternate isoforms of membrane proteins which would allow isolating and quantifying a cancer-specific subpopulation of vesicles.

Another object of this invention is to provide a method for identifying EV-specific tumor antigens.

It is advantageous to provide a method for identifying EV-specific tumor antigens based on initial small amounts of solid tumor cancer cells isolated from a patient and having a targeted phenotypic profile relevant to cancer progression.

Objects of this invention have been achieved by providing a method for the ex-vivo preparation of extracellular vesicles from cancer cells according to claim 1, ex-vivo generated extracellular vesicles according to claim 13 and uses thereof according to claim 14 and a method for identifying EV-specific tumor antigens, according to claim 15.

Disclosed herein is a method for the ex-vivo preparation of extracellular vesicles from cancer cells, said method comprising at least the steps of:
a) Providing a Schiff base cross-linking electrophilic substrate on the cell culture support;
b) Contacting the said Schiff base cross-linking electrophilic substrate with isolated solid tumor cancer cells in suspension in N-succinyl chitosan to trigger a Schiff base cross-linking reaction between the Schiff base cross-linking electrophilic substrate and the N-succinyl chitosan;
c) Adding a cell culture medium to the hydrogel resulting from the cross-linking reaction and leaving the hydrogel in this medium for about at least 24 hours;
d) Replacing the said culture medium with a new cell culture medium depleted of EVs;
e) Incubating the hydrogel containing cancer cells with the new cell culture medium depleted of EVs under conditions where cell-specific EVs are secreted;
f) Collecting and separating the cell-culture media from the cell-laden hydrogel.

Also disclosed herein are ex-vivo generated extracellular vesicles from solid tumor cancer cells, said ex-vivo generated extracellular vesicles expressing tumor specific antigens that are physiologically relevant to a specific type of cancer.

Also disclosed herein is a method of identifying EV-specific tumor antigens, said method comprising at least the steps of:
Providing ex-vivo generated EVs according to the invention;
Suspending said EVs at a concentration from 0.2-5.0 mg/mL in a lytic buffer;
Subjecting the lysate to an omics analysis;
Comparing the omics analysis profile of the ex-vivo generated EVs' profile with the omics analysis profile of healthy cells and/or cancer cells cultured in 2D;
Identifying differences in expression profile of the ex-vivo generated EVs.

In an advantageous embodiment, the methods of the invention and the ex-vivo generated extracellular vesicles allow the identification of a tumor antigen or a combination of tumor antigens specific to a certain type of solid cancer cells and/or cancer progression stage and which are useful in the development of diagnostic or therapeutic tools that are targeted to those.

### BRIEF DESCRIPTION OF THE DRAWINGS

FIG. 1 illustrates a method for obtaining ex-vivo extracellular vesicles from cancer cells where during culturing, the cells release extracellular vesicles into the medium.

FIG. 2 provides a comparison between material from extracellular vesicles obtained by two-dimensional (2D) cell culture system and by a method of the invention using a three-dimensional (3D) cell culture system as described in Example 1; A: Comparison of quantification of extracellular vesicular RNA of extracellular vesicles of the two systems normalized to the DNA content of the cells; B: Comparison of mass spectrometry analysis of cells cultured in a two-dimensional cell culture system (2D) compared to cells cultured in a three-dimensional cell culture system (3D) according to the invention.

FIG. 3 provides transmission electron microscopy (TEM) images of extracellular vesicles obtained by cell culture systems in 2D (A) and in 3D according to a method of the invention (B) (scale bars=200 nm).

FIG. 4 shows characterization of extracellular vesicles obtained by a method according to the invention by compared quantification of MHC II content of extracellular vesicles obtained in 3D according to a method of the invention with 2D cell culture systems.

FIG. 5 shows the rheological characterization of a hydrogel useful in a method of the invention as described in Example 3. A: The storage modulus of hydrogel systems with three different oxidized alginate (oxAlg) concentrations were assessed with frequency scans from 0.1-10 Hz. A minimum of 3 gels was measured per condition; B: Hydrogels in 24-well plates. Top: 3DB 6, middle 3DB 7.5, bottom 3DB 9. Scale bars 200 mm.

FIG. 6 shows the normalized exosome production as a function of oxidized alginate (oxAlg) concentration (w/v) as described in Example 3.

FIG. 7 shows clockwise from upper left: sChi with viscosities of 10, 20, 100, 500 mPa·s (resp., all 85% deacetylated) used to make microtissues containing MCF7 breast cancer cells as described in Example 3. Actin-rich cellular cytoskeletons stained with phalloidin (red) and nuclei stained with DAPI (blue). White triangles indicated extracellular vesicles (EVs) being secreted by cells in the 3D environment of the hydrogel. Scale bars=50 μm.

### DETAILED DESCRIPTION OF THE INVENTION

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group, which preferably consists only of these embodiments.

For the purposes of the present invention, the term “obtained” is considered to be a preferred embodiment of the term “obtainable”.

Where an indefinite or definite article is used when referring to a singular noun, e.g. “a”, “an” or “the”, this includes a plural of that noun unless something else is specifically stated. The terms “about” or “approximately” in the context of the present invention denote an interval of accuracy that the person skilled in the art will understand to still ensure the technical effect of the feature in question. The term typically indicates deviation from the indicated numerical value of ±20%, preferably ±15%, more preferably ±10%, and even more preferably ±5%.

The term “solid cancer cell” are cells from solid tumors.

The term “Schiff base cross-linking reaction between the Schiff base cross-linking electrophilic substrate and the N-succinyl chitosan” means that a Schiff base linkage occurs between complementary reactive groups of the Schiff base cross-linking electrophilic substrate and the N-succinyl chitosan, namely aldehyde/amine pairings. Schiff base cross-linking electrophilic substrate suitable according to the invention include any water-soluble polysaccharide that can be oxidized via reaction with sodium periodate. For example, a schiff base cross-linking electrophilic substrate according to the invention can be alginate, chondroitin sulfate or hyaluronic acid. Other examples of a schiff base cross-linking electrophilic substrate according to the invention include cellulose and glycogen.

The term “two-dimensional culture systems” refers to cell culture systems in which cells grow in a monolayer on a substrate are not encompassed by the term “three-dimensional cell culture system”. Two-dimensional cell culture system that may be used for comparison are standard polystyrene culture systems such as flat-bottom well plates or cell culture flasks comprising cells. The person skilled in the art is aware of methods for comparing antigen display of extracellular vesicles.

The term “lytic buffer” refers to a buffer capable to induce the lysis of the extracellular vesicles. Typically, a lytic buffer suitable in a method of the invention comprises 0.2% SDS (w/v).

The term “extracellular vesicle” refers to any membrane vesicles that a cell releases into the extracellular environment. Examples of extracellular vesicle are microvesicles and exosomes. Such extracellular vesicles typically have an average diameter of from about 10 nm to about 2,000 nm. In specific embodiments, the extracellular vesicles have an average diameter of from about 10 to about 250 nm, or alternatively from about 20 to about 200 nm, or alternatively from about 20 to about 175 nm, or alternatively from about 25 to 175 nm, or alternatively from about 40 to about 250 nm, or alternatively from about 40 to about 200 nm, or alternatively from about 50 to about 250 nm, or alternatively from about 50 to about 200 nm, or alternatively from about 50 to about 150 nm. It can be preferred that the extracellular vesicles have an average diameter from about 50 to about 150 nm.

The expression “omics analysis” intends to refers to the analysis and the characterization of specific classes of target biomolecules of cellular systems as a strategy to achieve comprehensive understanding of biological functions classical, therefore it would include the direct study of the proteome of EVs comprising the proteins, also called classical proteomics (e.g. by LC-MS/MS), but also other “omic” analyses such as the study of nucleic acids such as mRNA, miRNA, and 1ncRNA, also called transcriptomics (RNA sequencing) and the related lipids, also called lipidomics (e.g. LC ESI-MS/MS). According to a particular embodiment, the omics analysis is selected from a proteomics and a transcriptomics analysis or a combination of those.

The expression “tumor specific antigens that are physiologically relevant to a specific type of cancer” refers to molecular cargos expressed or carried in the EVs, such as proteins, nucleic acids, and lipids that are specific to a particular type of tumor. According to a particular aspect, those play a role in spreading of the disease and priming of the pre-metastatic niche.

The expression “culture medium depleted of EVs” refers to a culture medium from which most EVs have been removed (typically about 99% or more of the EVs are removed from the serum). Methods for depleting a culture medium from EVs are well known to a skilled person. Two main methods are used to deplete EVs from FBS: by ultracentrifugation of FBS (e.g. about 100,000 xg for 16 h) to pellet EVs, the pellet being then discarded or using centrifugal filters with 100,000 Dalton molecular weight cutoff to deplete EVs from FBS (e.g. commercially available from Centricon).

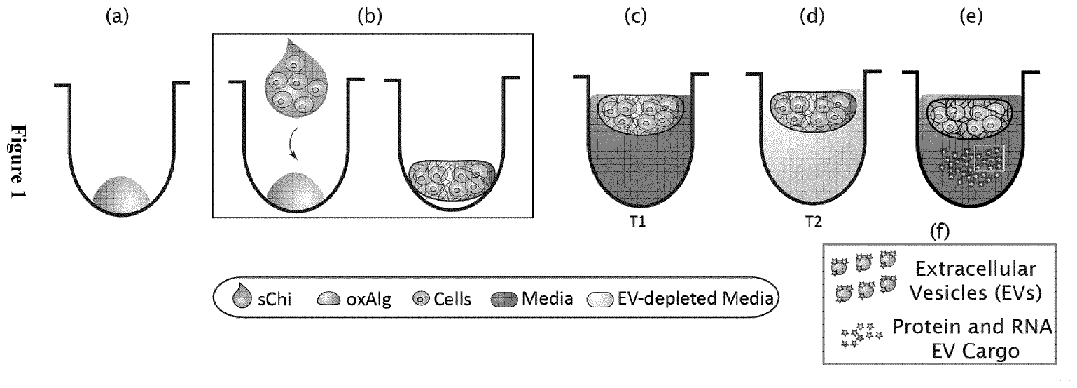

Referring to the figures, in particular first to FIG. 1, an illustration is provided of a method for the ex-vivo preparation of extracellular vesicles from cancer cells.

More specifically, the steps of the embodiment illustrated in FIG. 1 comprise:
a) Providing a Schiff base cross-linking electrophilic substrate on the cell culture support (e.g. oxidized alginate);
b) Contacting the said Schiff base cross-linking electrophilic substrate with isolated solid cancer cells in suspension in N-succinyl chitosan to trigger a Schiff base cross-linking reaction between the Schiff base cross-linking electrophilic substrate and the N-succinyl chitosan;
c) Adding a cell culture medium (e.g. standard culture medium containing fetal bovine serum (FBS) to the hydrogel resulting from the cross-linking reaction and leaving the hydrogel in this medium for about at least 24 hours (T1);
d) Replacing the said culture medium with a new cell culture medium depleted of EVs;
e) Incubating the hydrogel with the new cell culture medium depleted of EVs under conditions where cell-specific EVs are secreted;
f) Collecting and separating the cell-culture media from the cell-laden hydrogel.

According to another particular aspect, Schiff base cross-linking electrophilic substrate is oxidized alginate. Oxidized alginate can be obtained by oxidative cleavage of vicinal diols from alginate with suitable reagents such as sodium (meta) periodate and hydrogen peroxide, more particularly with sodium periodate for example such as described in Millan et al., 2015, Adv. Heathcare Mater., 4, 1348-1358 or Kowitsch et al, 2011, Biotechnol Appl Biochem, 58(5): 376-89. According to a particular embodiment, the degree of oxidation of hydroxyl groups lies within the range of about 25 to about 60%, for example within about 40 to about 55%, in particular within about 45 to about 50%.

According to a particular aspect, the method of the invention allows to modulate the yield of generated extracellular vesicles by modulating the stiffness of the hydrogel, in particular a stiffness mimicking more closely the microtumor environment.

According to a particular embodiment, a Schiff base cross-linking electrophilic substrate is oxidized alginate with oxidation degree from about 35 to about 45% and at concentration from about 1.0 to about 2.5% (w/v). Degree of oxidation can be determined by any person skilled in the art via elemental analysis following reaction with tert-butyl carbazate as described by Boonthekul et al., 2005, Biomaterials, 26(15): 2455.

According to another particular aspect, N-succinyl chitosan is obtained as described in Tan et al. 2009, Biomaterials, 30(13): 2499-506. N-succinyl chitosan is advantageously soluble at pH 7.4. N-succinyl chitosan suitable in a method of the invention bears enough of the amines so that they are available for Schiff base crosslinking. One preferred degree of substitution of amine groups of chitosan by succinyl groups lies within the range of about 25 to about 45%, preferably within about 30 to about 40% and more preferably about 35%.

According to another particular aspect, N-succinyl chitosan is a N-succinyl chitosan with about 85% deacetylation and viscosity of about 100-500 mPas, with N-substitution by succinic anhydride of about 30-40% and at a concentration of 0.8% (w/v) in PBS. N-substitution of chitosan can be determined by any person skilled in the art via nuclear magnetic resonance (1H-NMR) as described in Millan et al., 2015, Adv Healthec Mater, 4(9):1348.

According to another particular aspect, the Schiff base cross-linking electrophilic substrate is contacted with isolated solid cancer cells in suspension in N-succinyl chitosan to trigger the Schiff base cross-linking reaction for about 10 to about 35 min (e.g. 30 min) before adding the culture medium.

According to another particular aspect, the Schiff base cross-linking electrophilic substrate is contacted with isolated solid cancer cells in suspension in N-succinyl chitosan, wherein the cell density lies within the range of about 1×10⁶ to about 100×10⁶ cells/mL in the hydrogel resulting from the cross-linking reaction. Typically, the cell density lies within the range of about 2×10⁶ to about 20×10⁶ cells/mL in the hydrogel resulting from the cross-linking reaction.

According to another particular aspect, the hydrogel is incubated with the new cell culture medium depleted of EVs under conditions where cell-specific EVs are secreted for a time (T2) during which cell viability does not fall below about 90%. Viability can be measured according to widely used and commercially available viability assays (e.g. WST-1 or CellTiter Glo) at day 0 and then at every 3 days during the culture period. Normalization of viability to day 0 provides the % viability at each time point. Typically, the incubation time is from about 50 h to about 4 weeks, such as for about 70 h to about 2 weeks (e.g. about 72 h). In case of incubation time longer than about 3 days, the cells are cultured by repeating steps d) to f) to allow the cells to have a replenished nutrient supply, as illustrated in Example 2.

According to another particular aspect, solid cancer cells are selected from primary solid cancer cells and cells of solid cancer cell lines.

According to another particular aspect, solid cancer cells are from epithelial origin.

According to another further particular aspect, solid cancer cells are prostate cancer cells, non-small lung cancer cells, ovarian cancer cells, colorectal or gastrointestinal cancer cells, pancreatic cells, glioblastoma cells, breast cancer cells.

According to a particular aspect, the collected culture medium containing extracellular vesicles ex-vivo generated from solid cancer cells according to the invention is purified from large debris of cells or extracellular matrix (ECM) proteins, for example by centrifugation (e.g. 3′000 g) before further use.

According to another particular aspect, the collected culture medium containing extracellular vesicles ex-vivo generated from solid cancer cells according to the invention is isolated and concentrated to obtain a concentrated EV medium for further use, in particular in a method of identifying EV-specific tumor antigens. For example, centrifugal filters with a molecular weight cutoff can be used to concentrate the EVs in the medium. Typically, centrifugal filters with a 10 KDa molecular weight cutoff can be used. Typically, a concentrated EV medium contains EVs at a concentration from 0.2 to 5.0 mg/mL as determined as the total protein concentration in the solution of isolated EVs (e.g. as measured by Bio-Rad DC protein assay). Alternatively, the particle concentration can be determined by Nanoparticle Tracking Analysis and a concentrated EV medium contains EVs suitable for use in a method for identifying EV-specific tumor antigens according to the invention is from about 8.0×10¹⁰ to about 5×10¹² particles/mL.

According to another particular aspect, EVs are then extracted from the concentrated EV medium and separated from soluble proteins still present in the concentrated medium. Typically, size exclusion chromatography columns targeting particles with a size larger than 35 or 70 nm (e.g. 75 nm) can be used. The 35 nm columns typically isolate particles between 35 and 350 nm and the 70 nm columns isolate between 70 and 1,000 nm.

According to another particular aspect, the EVs are sometimes too dilute in the buffer after size exclusion chromatography for a direct use in a method according to the invention. To reconcentrate them, the purified and extracted EVs are first pelleted via ultracentrifugation (e.g. 120′000 g for 2 h) and then resuspended in a smaller volume of PBS. They can then be stored at about −80° C. for up to 6 months before further use.

According to a particular aspect, are provided ex-vivo generated extracellular vesicles from cancer cells of solid tumors obtainable from a method according to the invention.

According to a particular aspect, said ex-vivo generated extracellular vesicles from cancer cells of solid tumors according to the invention express tumor specific antigens that are physiologically relevant to a specific type of cancer and the number and level of unique tumor specific antigens expressed in those ex-vivo generated extracellular vesicles is higher in number compared to the EVs from cells cultured in 2D.

According to a further particular aspect, ex-vivo generated extracellular vesicles from solid cancer cells according to the invention present a number of unique proteins identified in proteomics analysis according to the invention about 20-55% higher than in EVs from 2D cultures. This is due to low-intensity proteins (i.e. low abundance) in 2D samples not surpassing the limit of detection (LOD) of LC-MS/MS, while the levels of those are higher in ex-vivo generated extracellular vesicles from solid cancer cells according to the invention.

Ex-vivo generated extracellular vesicles according to the invention, wherein said extracellular vesicles allow for an increased antigen display of tumor antigens, for example as characterized in a method according to the invention, compared to extracellular vesicles obtained by a method using a two-dimensional cell culture system.

According to a particular aspect, the said antigen is a human membrane protein.

According to a particular aspect, the said antigen is selected from the group consisting of ion channels, G-protein coupled receptors, multispanning intramembrane targets implicated in signaling pathways associated with disease and antigen targets associated with autoimmune diseases.

According to a particular aspect, some ex-vivo generated extracellular vesicles from solid cancer cells according to the invention express alternate isoforms of membrane proteins that are cancer-specific and those would allow isolating and quantifying a cancer-specific subpopulation of vesicles.

According to a particular aspect, some ex-vivo generated extracellular vesicles from solid cancer cells according to the invention secrete full length proteins that are not expressed by corresponding normal or healthy cells.

According to a particular aspect, some ex-vivo generated extracellular vesicles from solid cancer cells according to the invention secrete proteins with different methylation/phosphorylation of proteins compared to corresponding normal or healthy cells that alter their activity.

A further specific embodiment relates to ex-vivo generated extracellular vesicles as described herein for use in identifying extracellular vesicular tumor antigens. In a specific embodiment, said extracellular vesicles allow for an increased antigen display of tumor antigens compared to extracellular vesicles obtained by a method using a two-dimensional cell culture system.

According to another particular aspect, ex-vivo generated extracellular vesicles obtained according to a method of the invention present a higher abundance of tumor antigens, such extracellular vesicles may be used to identify extracellular vesicular tumor antigens. For instance, extracellular vesicles that are obtained according to the methods of the present invention may be used to identify novel extracellular vesicular tumor antigens.

According to a particular aspect, the method of the invention allows to create a culture medium that is physiologically relevant to a micro-tumor environment. The use of non-mammalian polysaccharide for the formation of the hydrogel seems to be of importance to preserve a physiologically relevant behavior and phenotype.

According to a particular aspect, the method of the invention allows to create a culture medium that allows the secretion and adsorption of the secreted proteins to the extracellular environment and create a favorable ECM which is critical for the production of EVs.

According to another aspect, those ex-vivo generated extracellular vesicles can be used in a method for identifying EV-specific tumor antigens.

According to another further aspect, is provided a method of identifying EV-specific tumor antigens, said method comprising at least the steps of:
Providing ex-vivo generated EVs according to the invention;
Suspending said EVs at a concentration from about 0.2-5.0 mg/mL in a lytic buffer;
Subjecting the lysate to an omics analysis, in particular a tandem LC/MS analysis;
Comparing the omics analysis profile, in particular the LC/MS profile of the ex-vivo generated EVs' profile with the omics analysis profile, in particular the LC/MS profile of healthy cells and/or cancer cells cultured in 2D;
Identifying differences in expression profile of the ex-vivo generated EVs.

According to particular aspect, ex-vivo generated extracellular vesicles obtained according to the methods of the present invention differ in properties compared to extracellular vesicles obtained by a method using a two-dimensional cell culture system, for instance, an increased tumor antigen display.

According to particular aspect, tumor antigens are overexpressed when cells were cultured according to a method of the invention. Thus, the secreted extracellular vesicles are suggested to show increased antigen display for the overexpressed tumor antigens.

An increased antigen display may result from a higher in number of extracellular vesicles meaning that more extracellular vesicles may be obtained from the same number of cells if the cells are cultured according to the methods of the present invention (compared to cells cultured in a two-dimensional cell culture system). Further, if the same number of extracellular vesicles is compared, increased antigen display may result from the different properties that are provided by extracellular vesicles obtained by the methods of the present invention (if compared to extracellular vesicles obtained by a two-dimensional cell culture system).

### EXAMPLES

### Example 1—Obtaining Ex-Vivo Extracellular Vesicles Released by Melanoma Cells and Ovarian Cancer Cells

Ex vivo extracellular vesicles released by melanoma cells cultured were obtained according to a method of the invention and as illustrated on FIG. 1 and compared with extracellular vesicles released by melanoma cells cultured in a two-dimensional cell culture system as described below.

a) Providing a Schiff Base Cross-Linking Electrophilic Substrate on the Cell Culture Support (e.g. Oxidized Alginate)

Sodium alginate was purchased from Kimica (Tokyo, Japan) and its oxidation was carried out with sodium periodate (Sigma, Buchs, Switzerland) as described in Köwitsch et al, 2011, supra. The oxidized alginate was then lyophilized after reaction and then solubilized at a concentration of 10 mg/mL in PBS. Drops of 10 μL each of oxidized alginate (oxAlg, 10 mg/mL, 50% degree of substitution) were then placed in individual wells of U-bottom plates.

b) Contacting the Said Schiff Base Cross-Linking Electrophilic Substrate with Isolated Solid Cancer Cells in Suspension in N-Succinyl Chitosan to Trigger a Schiff Base Cross-Linking Reaction Between the Schiff Base Cross-Linking Electrophilic Substrate and the N-Succinyl Chitosan

Chitosan, deacetylation degree of 85%, viscosity of 500 mPa's was purchased from Heppe Medical Chitosan GmbH (Halle, Germany) and was succinylated by reacting succinic anhydride (Sigma) with the chitosan as described in Tan et al., 2009, supra. The resulting succinate-modified chitosan was then lyophilized and solubilized in PBS to a concentration at 8 mg/mL. B16F10 melanoma cells were suspended in the solution of 8 mg/ml succinate-modified chitosan (sChi, 35% degree of substitution) at a cell density of 20×10⁶ cells/mL. 5 μL of this mixture were pipetted into individual droplets of oxAlg. The reaction was carried out in an incubator at 37ºC for about 30 minutes.

c) Adding a Cell Culture Medium to the Hydrogel Resulting from the Cross-Linking Reaction and Incubating the Cells for about 24 Hours

After incubation, medium was added to the wells containing DMEM, 10% (v/v) fetal bovine serum and 1% penicillin-streptomycin for about 3-4 days.

d) Replacing the Said Culture Medium with a New Cell Culture Medium Depleted of EVs

(e.g. FBS that has been depleted from EVs e.g. by centrifugal filters with 100,000 Dalton molecular weight cutoff);

e) Incubating the Hydrogel with the New Cell Culture Medium Depleted of EVs Under Conditions where Cell-Specific EVs are Secreted;

The cell culture system in DMEM, 10% (v/v) fetal bovine serum depleted of exosomes, and 1% penicillin-streptomycin.

f) Collecting and Isolating the Cell-Culture Medium Containing the Secreted EVs from the Hydrogel.

Steps d) to f) was repeated roughly every 3-days during about 14 days in order to compare EV production ability with 2D systems. Alternatively, the cells can be cultured without repeating steps d) to f) as described in Example 3 if culture is carried out for less than 3 days.

For comparison, B16F10 melanoma cells were also cultured in polystyrene flat-bottom well plates for 14 days (two-dimensional cell culture systems). Cells were seeded at an initial density of 5,000/cm² in cell culture media containing DMEM, 10% (v/v) fetal bovine serum and 1% penicillin-streptomycin. After 24 h, the media was replaced by a cell culture media depleted of EVs. About every 3 days the media containing EVs secreted by cells grown in 2D was collected and replaced with fresh media depleted of EVs. This was repeated for 14 days.

Ex vivo extracellular vesicles released by SKOV-3 cells (human ovarian cancer cell line) were prepared in the same way. The cell culture systems comprising SKOV-3 cells were cultured for 4 days in DMEM, 10% (v/v) fetal bovine serum depleted of exosomes, and 1% penicillin-streptomycin. For comparison, SKOV-3 cells were also cultured in polystyrene flat-bottom well plates for 4 days (two-dimensional cell culture systems). Cells were lysed after the culture period and total protein was harvested and analyzed with mass spectrometry.

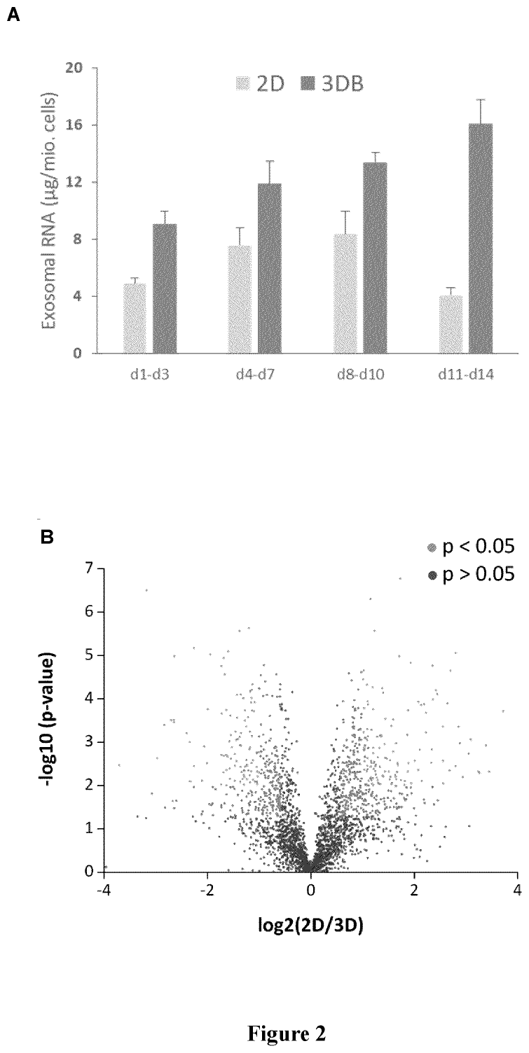

### Quantification of Extracellular Vesicular RNA for the B16F10 Melanoma Cells (FIG. 2A)

Cells secreted EVs from day 0 to day 3, media was collected and replaced with fresh EV-free media, cells secreted EVs from day 3-day 7, etc. For each batch of 3 days, the amount of EVs produced in the culture media from each of the culture systems were compared (of the invention and 2D-comparative culture system). In 3D culture system according to the invention, the cells increase production over 2 weeks, while in 2D, the amount of EV production eventually drops off. The EVs were harvested during the indicated time (e.g. day 1-day 3) and the extracellular vesicles that had been secreted into the media during that time period were harvested via ultra-centrifugation according to Hoshino et al., 2015, Nature 527, 329-335. Extracellular vesicles were then lysed and the total RNA was quantified. At the same time, the number of cells was quantified by an assay measuring double stranded DNA (PicoGreen Assay). FIG. 2A represents extracellular vesicular RNA normalized to DNA content which gives the extracellular vesicle number normalized to the number of cells. It is clear from this figure that in a method of the invention, cells produce significantly more extracellular vesicles for a much longer period of time. This means higher extracellular vesicles concentrations can be achieved (FIG. 2A) by a method of the invention.

Mass spectrometry analysis of total cell protein for SKOV3 cells (FIG. 2B)

The ‘volcano plot’ in FIG. 2B shows the difference in abundance of each identified protein between the method of the invention and 2D culture. To the right of ‘0’ on the x-axis means that the protein was overexpressed by the method of the invention, to the left it means the protein was expressed less in the system of the invention. The y-axis is the confidence value in that measured difference. The lighter grey dots indicate proteins differentially expressed by +/−1.5x with a p<0.05. It is clear from this figure that a number of tumor antigens were overexpressed in the system of the invention. If the cell has higher abundance of a given protein, it follows that the secreted extracellular vesicles will also have higher probability to be enriched with that protein.

Similar findings were observed with B16F10 cells where 10.92% of the identified proteins were changed with EVs obtained by a method according to the invention more than 2-fold with a p-value less than 0.01.

Further, due to the higher abundance of tumor antigens in cancer cells cultured in a method according to the invention, extracellular vesicles secreted from such cells may be used to identify (novel) extracellular vesicular tumor antigens.

A list of known tumor-associated antigens has been published by Gires and Seliger, 2009, John Wiley & Sons. With the method of identifying EV-specific tumor antigens according to the invention, the proteomics analysis led to a protein profile that was found to be significantly enriched in SKOV3 cells cultured according to a method of the invention and among the enriched protein profile, some of those match the list of known tumor-associated antigens which includes the following: LPLAT 7 (Lysophospholipid acyltransferase 7, bladder/breast, UniProt Accession No. Q96N66, SEQ ID NO: 1); NY-REN-49 (Mitochondrial RNase P protein 1, renal carc. antigen, UniProt Accession No. Q7L0Y3, SEQ ID NO: 2); NY-REN-39 (Succinate-CoA ligase [ADP-forming] subunit beta, renal carc. antigen, UniProt Accession No. Q9P2R7, SEQ ID NO: 3); NY-REN-42 (Alanine-tRNA ligase, renal carc. antigen, UniProt Accession No. P49588, SEQ ID NO: 4); Titin (Rhabdomyosarcoma antigen MU-RMS-40.14, UniProt Accession No. Q8WZ42, SEQ ID NO: 5); Carbonic anhydrase 12 (Tumor antigen HOM-RCC-3.1.3, UniProt Accession No. 043570, SEQ ID NO: 6); Dipeptidyl peptidase 4 (CD26; T-cell activation antigen, UniProt Accession No. P27487, SEQ ID NO: 7); Galectin-3-binding protein (Tumor-associated antigen 90K, UniProt Accession No. Q08380, SEQ ID NO: 8); CD63 antigen (Ocular melanoma-associated antigen, UniProt Accession No. P08962, SEQ ID NO: 9) and HLA class I histocompatibility antigen, B-35 alpha chain (UniProt Accession No. P30685, SEQ ID NO: 10).

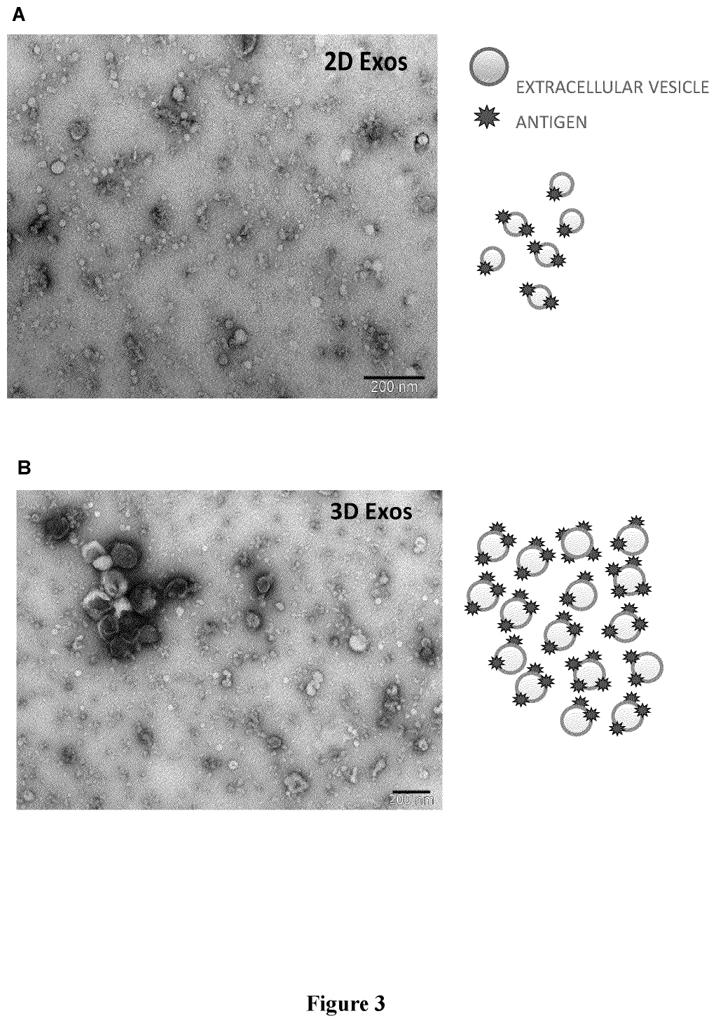

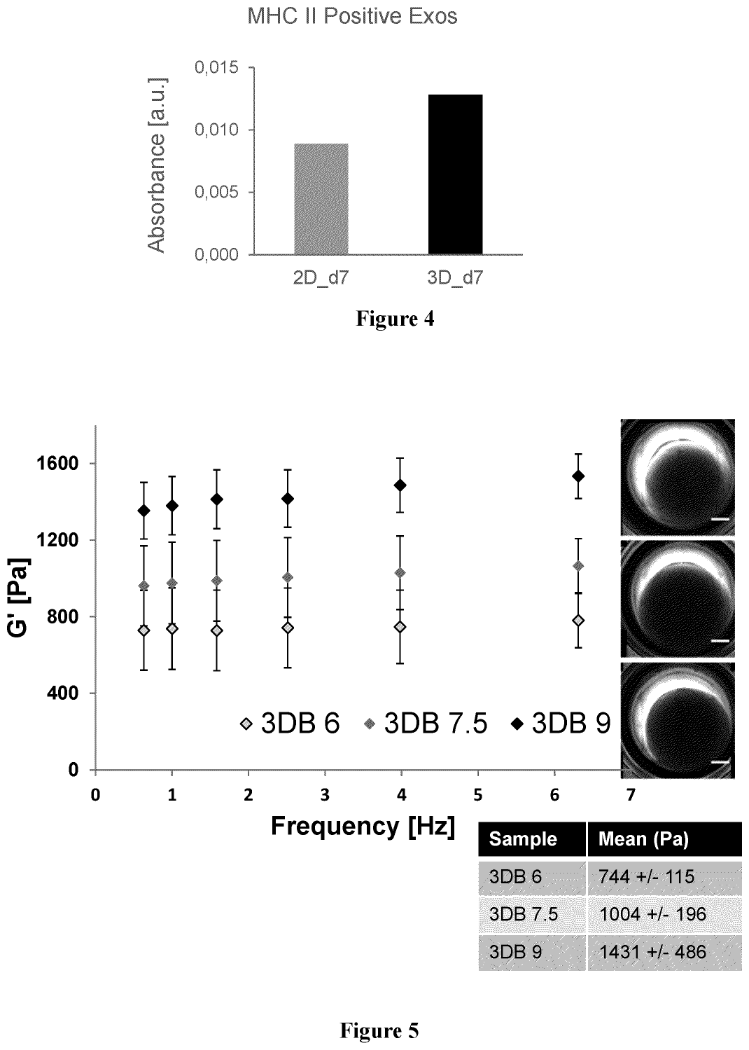

### Transmission Electron Microscopy of Extracellular Vesicles (FIG. 3)

Quantification of extracellular vesicular RNA of the EVs obtained according to a method of the invention or in a 2D system was carried out via ultracentrifugation was used to perform transmission electron microscopy (TEM) imaging to produce the images of FIG. 3. 30 μL aliquots were allowed to adsorb on formvar/carbon-coated grids for 15 minutes, which were then washed three times with PBS. The samples were then negatively stained in 2% uranyl acetate and thereafter examined with a Tecnai F30 microscope (FEI). The images show a higher density of vesicles in the preparation by a method of the invention as well as a higher number of ‘larger’ (i.e. diameter of approx. 100 nm) vesicles. Those larger diameter values are consistent with literature reports of the specific class of extracellular vesicles known as exosomes.

### Example 2—Use of EVs of the Invention for Biomarker Discovery

EVs are prepared according to a method of the invention as follows:

Oxidized alginate (39% degree of oxidation) is solubilized in sterile PBS at 1.0% (w/v). N-succinyl chitosan (85% degree of deacetylation, 500 mPas viscosity, 35% N-substitution) is solubilized in sterile PBS at 0.8% (w/v). 20 μl droplets of oxidized alginate are pipetted to the wells of U-bottom 96-well plates. 144×10⁶ LNCaP prostate cancer cells are resuspended in 7.2 ml of N-succinyl chitosan and added to the oxidized alginate in 5 μl droplets per well. After adding cells, the well plates are incubated at 37ºC for 15-35 minutes, after which 150 μl media containing RPMI (Roswell Park Memorial Institute medium), 10% fetal bovine serum, and 1% penicillin/streptomycin is added to each well. The well plates are stored for 24 h at 37° C. in a humidified incubator with 5% CO₂. After 24 h, the media is removed via pipette and discarded. Media containing RPMI, 10% fetal bovine serum depleted of EVs, and 1% penicillin-streptomycin is added to each well in a volume of 150 μl/well and the cells are incubated as before for a further 72 h. After 72 h, media containing EVs secreted by cells cultured according to the invention is collected via pipette and pooled. Well plates containing cells are discarded. In parallel, LNCaP prostate cancer cells are cultured in 2D in 20×500 cm² (at a confluency of approximately 20,000 cells/cm²) tissue culture plates.

The media is aspirated and changed to EV-depleted media as described above for 3D cultures. After 72 h, media containing EVs secreted by cells cultured in 2D is collected and pooled, and culture plates are discarded.

Pooled media is then centrifuged at 3,000×g for 10 minutes at 4° C. to pellet cell debris. Supernatant is transferred to clean/sterile bottles and pellets are discarded. Media cleared of cell debris is then concentrated about 150× in centrifugal filters with 10,000 Dalton molecular weight cutoff (Centricon). EVs are then isolated from concentrated media via size exclusion chromatography using qEV columns with 70 nm cutoff (Izon Biosciences).

The obtained Evs are then prepared to be subjected a method of identifying EV-specific tumor antigens according to the invention as follows:

### Providing Ex-Vivo Generated EV's According to the Invention

EV-containing fractions (F6-F10) are collected, pooled, and pelleted via ultracentrifugation (100,000×g, 2 h, 4° C.).

Suspending Said EV's at a Concentration from about 0.2-5.0 mg/ml. In a Lytic Buffer

Supernatant is carefully discarded and the EV pellet is resuspended in lysis buffer (“Lyse” from the commercial iST Kit from PreOmics). Protein concentration is measured via Qubit protein assay according to manufacturer's protocol (ThermoFisher). 50 μg of protein per sample is digested on the membrane by adding 50 μl of the ‘Digest’ solution. After 60 minutes of incubation at 37° C., the digestion was stopped with 100 μl of Stop solution. The samples are then centrifuged and the supernatant transferred to the cartridge. The solutions in the cartridge are removed by centrifugation at 3,800×g, while the peptides are retained by the iST-filter. Finally, the peptides are washed, eluted, dried and re-solubilized in 20 μL of injection buffer (3% acetonitrile, 0.1% formic acid).

### Subjecting the Lysate to a Proteomics Analysis, in Particular a Tandem LC MS Analysis

Mass spectrometry analysis is performed on a QExactive mass spectrometer coupled to a nano EasyLC 1000 (Thermo Fisher Scientific) as described below. Proteomics analysis of data is carried out to determine proteins that are enriched or exclusively present in EVs from LNCaP cells cultured in a method of the invention.

Solvent composition at the two channels is 0.1% formic acid for channel A and 0.1% formic acid, 99.9% acetonitrile for channel B. For each sample 4 μL of peptides is trapped on an Acclaim PepMap 100 trap column (3 μm, 75 μm×2 cm, C18, 100 Å, Thermo Fisher Scientific) and separated on an EASY-Spray C18 column (2 μm, 75 μm×50 cm, 100 Å, Thermo Fisher Scientific) at a flow rate of 300 nL/min by a gradient from 5 to 22% B in 30 min, 32% B in 10 min and 95% B in 10 min. The mass spectrometer is operated in data-dependent mode (DDA), acquiring a full-scan MS spectra (300-1,700 m/z) at a resolution of 70,000 at 200 m/z after accumulation to a target value of 3,000,000 followed by HCD (higher-energy collision dissociation) fragmentation on the twelve most intense signals per cycle. HCD spectra is acquired at a resolution of 35,000 using a normalized collision energy of 25 and a maximum injection time of 120 ms. The automatic gain control (AGC) is set to 50,000 ions. Charge state screening was enabled and singly and unassigned charge states were rejected. Only precursors with intensity above 8,300 are selected for MS/MS (2% underfill ratio). Precursor masses previously selected for MS/MS measurement are excluded from further selection for 30 s, and the exclusion window is set at 10 ppm. The samples are acquired using internal lock mass calibration on m/z 371.1010 and 445.1200.
Comparing the proteomics analysis profile, in particular the LC/MS profile of the ex-vivo generated EVs' profile with the proteomics analysis profile, in particular the LC/MS profile of healthy cells and/or cancer cells cultured in 2D; and
Identifying differences in expression profile of the ex-vivo generated EVs.

A list of biomarker ‘hits’ is then generated based on increased abundance in EVs from cancer cells cultured in a method of the invention compared to cancer cells grown in 2D or healthy cells grown in 2D or 3D.

### Example 3—Modulation of the Rheological Properties of the Hydrogels for Use in a Method of the Invention

Hydrogels formed by Schiff base cross-linking reaction between N-succinyl chitosan (sChi) containing cells and oxidized alginate (oxAlg) according to a method of the invention have been referred to as 3DBs. The modulation of the properties of those hydrogels has been studied as a function of the concentration of oxidized alginate (oxAlg) and the chitosan chain length.

Effect of the Concentration of Oxidized Alginate (oxAlg)

The rheological properties of those hydrogels can be modulated by increasing the concentration of the oxidized oxAlg which results in the formation of stiffer hydrogels. As an example, hydrogels with 6, 7.5, or 9 mg/mL oxAlg (3DB 6, 3DB 7.5, or 3DB 9, respectively) were formed with sChi of 8 mg/mL as described herein. The stiffness of these 3DB hydrogels was quantified using rheology with constant 1% strain and frequency scans from 0.1-10 Hz at 37° C. Increasing G′ values at increasing oxAlg concentration shows that stiffness correlates with oxAlg concentration (FIG. 5). This modularity is important as it has been shown that tumor tissue is stiffer than healthy tissue (Paszek et al., 2005, Cancer cell, 8, 241) and therefore, it is advantageous to be able to control the stiffness of 3D hydrogels to simulate a physiologically relevant environment.

B16F10 murine melanoma cells were seeded in hydrogels with oxAlg concentrations ranging from 0.5% to 2% (w/v in PBS) according to a method of the invention. Secreted exosomes were isolated from conditioned media and the total amount of exosomal RNA present was quantified by UV/VIS spectrophotometry (NanoDrop) as described above. The quantity of exosome-secreting cells in each condition was assessed by PicoGreen assay (ThermoFisher) for double stranded DNA. RNA amount is depicted above as normalized to cell number.

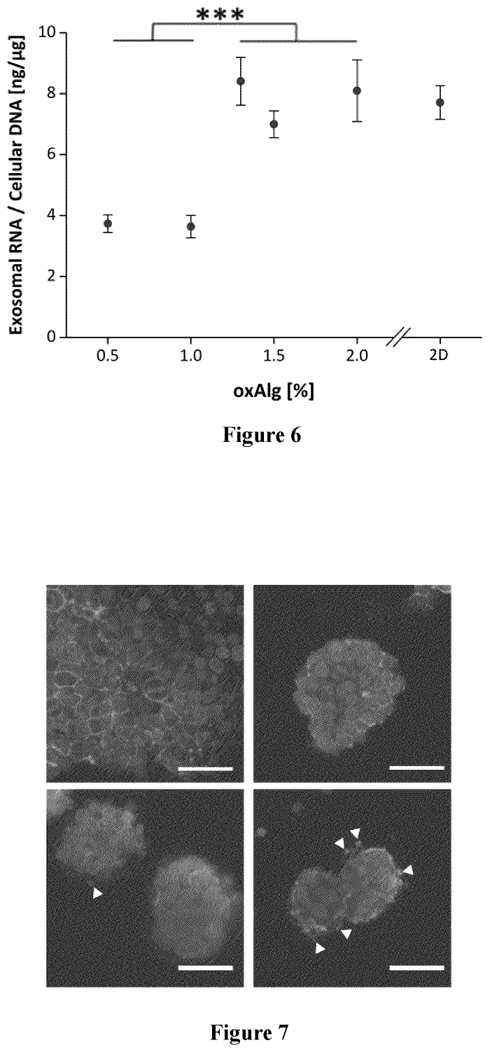

After only 3 days in culture, cells grown in hydrogels with a relatively high concentration of oxAlg (2% w/v) released 2.26 times more exosomes than cells seeded in gels made with low concentrations of oxAlg (0.5% to 1% w/v, FIG. 6). To assess the statistical significance despite the low sample number of the exosome isolates, the sigmoidal-like distribution values were used. The samples were gathered in two groups (0.5%-1% w/v and 1.3-2% w/v) and the significance of the difference between those two groups was assessed by a two-sided t-test, which was attested a high significance (***p<0.01). In the isolate of 2D grown cells, a very comparable amount of RNA was measured as in cells grown in 2% oxAlg hydrogels.

This data supports the influence of the stiffness of the polymer matrix on exosome secretion, and that this can be controlled by altering the concentration of oxidized alginate used to form the hydrogel.

### Effect of the Chitosan Chain Length

Various chitosan having various viscosity in solution were tested. More precisely, the viscosity of a 1% (w/v) solution in a 1% (v/v) acetic acid solution at 20° C. is listed, which is an indirect measure of chain length since chitosan chain length correlates with its viscosity in solution (Costa et al., 2015, Carbohydrate polymers 2015, 133, 245)

N-succinylated chitosan (sChi) were prepared as described herein using chitosan viscosities of 10, 20, 100, and 500 mPas, all having the same degree of deacetylation (85%) to isolate chitosan chain length as a variable. MCF7 breast cancer cells were encapsulated in hydrogels composed of sChi (0.8%, w/v) and oxidized alginate (1.0% w/v) by Schiff base cross-linking reaction between N-succinyl chitosan (sChi) containing cells and oxidized alginate (oxAlg) according to a method of the invention. After 4 days in culture, cells in hydrogels were fixed and stained with phalloidin (red) to visualize cytoskeleton and DAPI to visualize cell nuclei (blue). It was then observed that higher viscosity sChi resulted in smaller microtumors which were more likely to secrete actin-rich extracellular vesicles (indicated by white triangle), which is more favorable for biomarker identifications using EVs according to the invention (FIG. 7).

1-15. (canceled)

16. A method for the ex-vivo preparation of extracellular vesicles from cancer cells, said method comprising at least the steps of:
a) providing a Schiff base cross-linking electrophilic substrate on the cell culture support;
b) contacting the said Schiff base cross-linking electrophilic substrate with isolated solid cancer cells in suspension in N-succinyl chitosan to trigger a Schiff base cross-linking reaction between the Schiff base cross-linking electrophilic substrate and the N-succinyl chitosan;
c) adding a cell culture medium to the hydrogel resulting from the cross-linking reaction and leaving the hydrogel in this medium for about at least 24 hours;
d) replacing the said culture medium with a new cell culture medium depleted of EVs;
e) incubating the hydrogel with the new cell culture medium depleted of EVs under conditions where cell-specific Evs are secreted; and
f) collecting and separating the cell-culture media from the cell-laden hydrogel.

17. The method according to claim 16, wherein the Schiff base cross-linking electrophilic substrate is oxidized alginate.

18. The method according to claim 16, wherein the Schiff base cross-linking electrophilic substrate is contacted with isolated solid cancer cells in suspension in N-succinyl chitosan to trigger the Schiff base cross-linking reaction for about 10 to about 35 min before adding the culture medium.

19. The method according to claim 16, wherein oxidized alginate with oxidation degree from about 35% to about 45% and at concentration from about 1.0% to about 2.5% (w/v) is used as a Schiff base cross-linking electrophilic substrate and a N-succinyl chitosan with about 85% deacetylation and viscosity of about 100-500 mPas, with N-substitution by succinic anhydride of about 30%-40% at a concentration of 0.8% (w/v) in PBS is used.

20. The method according to claim 16, wherein the Schiff base cross-linking electrophilic substrate is contacted with isolated solid cancer cells in suspension in N-succinyl chitosan, wherein the cell density lies within the range of about 1×10⁶ to about 100×10⁶ cells/mL in the hydrogel resulting from the cross-linking reaction.

21. The method according to claim 16, wherein the Schiff base cross-linking electrophilic substrate is contacted with isolated solid cancer cells in suspension in N-succinyl chitosan, wherein the cell density lies within the range of about 2×10⁶ to about 20×10⁶ cells/mL in the hydrogel resulting from the cross-linking reaction.

22. The method according to claim 16, wherein solid cancer cells are selected from primary solid cancer cells and cells of solid cancer cell lines.

23. The method according to claim 16, wherein solid cancer cells are from epithelial origin.

24. The method according to claim 16, wherein solid cancer cells are prostate cancer cells, non-small lung cancer cells, ovarian cancer cells, colorectal or gastrointestinal cancer cells, pancreatic cells, glioblastoma cells, breast cancer cells.

25. The method according to claim 16, wherein the collected culture medium containing extracellular vesicles ex-vivo generated from solid cancer cells is purified from large debris of cells or extracellular matrix (ECM) proteins.

26. The method according to claim 16, wherein the collected culture medium containing extracellular vesicles ex-vivo generated from solid cancer cells is isolated and concentrated to obtain a concentrated EV medium.

27. The method according to claim 26, wherein, EVs are then extracted from the concentrated EV medium and separated from soluble proteins.

28. Ex-vivo generated extracellular vesicles from solid cancer cells obtainable from a method according to claim 16, wherein the number and the abundance of tumor specific antigens expressed in those ex-vivo generated extracellular vesicles is higher in number compared to the EVs from cells cultured in 2D.

29. A method of identifying EV-specific tumor antigens, said method comprising at least the steps of:
providing ex-vivo generated EVs according to a method according to claim 16;
suspending said EVs at a concentration from 0.2 to 5.0 mg/mL in a lytic buffer;
subjecting the lysate to an omics analysis, wherein said omics analysis is selected from a proteomics and a transcriptomics analysis or a combination or proteomics and transcriptomics analysis;
comparing the omics analysis profile of the ex-vivo generated EVs' profile with the omics analysis profile of healthy cells and/or cancer cells cultured in 2D; and
identifying differences in expression profile of the ex-vivo generated EVs.
